(19) United States
(12) Patent Application Publication
(10) Pub. No.: US 2011/0203590 A1
(43) Pub. Date: Aug. 25, 2011

(54) DIRECTIONAL VALVE FOR A RESPIRATOR PRODUCT

(75) Inventors: Jürgen UNGER, Berlin (DE); Alexander HEIMANN, Nehms (DE); Jörn HIRSCHBIEGEL, Lubeck (DE); Jörg POLZIEN, Ratzeburg (DE)

(73) Assignee: Drager Safety AG & Co. KGaA, Lubeck (DE)

(21) Appl. No.: 12/963,810

(22) Filed: Dec. 9, 2010

(30) Foreign Application Priority Data
Feb. 23, 2010 (DE) 10 2010 008 923.0

(51) Int. Cl. A61M 16/20

(52) U.S. Cl. 128/205.24

(57) ABSTRACT

A directional valve for a respirator product shall be improved with regard to a low flow resistance. To accomplish the object, two diaphragm-like valve disks (12, 13) abutting against one another at a central line of separation (16) that are attached to the valve housing (11) in a point-like manner and can be moved in a flap-like manner by the breathing gas stream are provided. A central web (19) at the valve housing (11), which runs along the line of separation (16) and support webs (21) additionally arranged on both sides, is used as a valve seat.

### CROSS REFERENCE TO RELATED APPLICATIONS

This application claims the benefit of priority under 35 U.S.C. §119 of German Patent Application DE 10 2010 008 923.0 filed Feb. 23, 2010, the entire contents of which are incorporated herein by reference.

### FIELD OF THE INVENTION

The present invention pertains to a directional valve for a respirator product.

### BACKGROUND OF THE INVENTION

A directional valve of the type mentioned in the form of an exhalation valve on a respirator mask has become known from DE 10 27 518. The directional valve consists of a valve lower part with a valve seat and a closing element held in the center by a web. To limit the lateral forces developing during deformation of the closing element, the closing element has a truncated-cone-shaped design and has disk-shaped sections offset against one another in a step-like manner. The drawback of the prior-art directional valve is that in the direction of flow through the closing element, only a part of the cross-sectional area is released, and, if the directional valve is within a breathing tube, the breathing gas is deflected through the closing element towards the tube wall, which increases the flow resistance. In the use of directional valves in closed-circuit respirators, low flow resistances are required in order to limit the breathing effort of the user of the device to a minimum.

Closed-circuit respirators supply a user of the device with breathing gas when work has to be done in an environmental atmosphere with toxic gases. Within the closed-circuit respirator the breathing gas is guided in the closed circuit, wherein exhaled carbon dioxide is removed and then consumed breathing gas is replaced. To achieve a directed breathing gas transport within the breathing closed circuit, directional valves are provided both in the inhalation tube and in the exhalation tube. A closed-circuit respirator of the type mentioned is disclosed in, for example, DE 39 30 362 C2.

### SUMMARY OF THE INVENTION

The basic object of the present invention is to improve a directional valve of the type mentioned with regard to a low flow resistance.

According to the invention, a directional valve for a respirator product is provided with an inner area and an outer area, a ring-shaped valve housing in the outer area and two diaphragm-like valve disks abutting against one another at a line of separation, which valve disks have each a first section fixed at the valve housing and a movable second section running towards the line of separation. The valve housing has a central web as a valve seat running along the line of separation and support webs arranged on both sides of the central web. The valve disks are designed as resting on the central web and the support webs in the locking direction and as removable in a flap-like manner by the breathing gas stream in the passing direction.

The valve disks may advantageously consist of disk-shaped rubber or elastomer material, and preferably of silicone rubber. The valve disks may have an average thickness between 0.6 mm and 1.2 mm.

The inside diameter of the valve housing may advantageously be between 35 mm and 50 mm. The valve disks may have a Shore hardness of 20° Sha to 30° Sha.

The directional valve according to the present invention has two valve disks abutting against one another at a line of separation, which have a semicircular design, wherein the line of separation is the axis of symmetry of the valve disks. The valve disks consist of thin, flexible elastomer material and are each attached in a punctiform manner in a first section at a valve housing with a ring-shaped design. The valve disks cover the inside cross-sectional area of the valve housing. In a second section adjacent to the first section, which runs up to the line of separation, the valve disks are freely movable. As a contact surface for the valve disks, the valve housing has a central web running along the line of separation, and support webs are additionally arranged on both sides of the central web. In the locking direction of the directional valve, the valve disks lie on the central web and the support webs, and in the passing direction, they are opened in a flap-like manner by the breathing gas stream. If the directional valve is arranged in a breathing tube, the valve disks lie against the inner wall of the breathing tube in the passing direction, and the breathing gas can flow freely through the valve housing without the gas stream being deflected or obstructed in any way by the valve disks. The punctiform attachment of the valve disks in the first section additionally brings about that the valve disks are able to move in the breathing gas stream without greater restoring forces.

The valve disks may comprise thin rubber or elastomer material, and preferably of silicone rubber. The average thickness of the valve disks is between 0.6 mm and 1.2 mm; a preferred thickness is 0.8 mm. To achieve a good flow of the valve housing, its inside diameter is between 35 mm and 50 mm, the preferred diameter is 40 mm. The valve disks have a Shore hardness between 20° Sha and 30° Sha.

An exemplary embodiment of the directional valve according to the present invention is shown in the figures and explained below in greater detail. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a sectional view showing a first directional valve according to the state of the art;

FIG. 2 is a perspective sectional view showing a directional valve according to the present invention;

FIG. 3 is a perspective view showing the directional valve according to FIG. 2 in the flow direction;

FIG. 4 is a perspective view showing the directional valve according to FIG. 2 in the locking direction;

FIG. 5 is a schematic view showing an arrangement for testing a respirator;

FIG. 6 is a view showing measurement curves for the directional valve according to the present invention and a directional valve according to the state of the art, for a respiratory minute volume of 50 L; and

FIG. 7 is a view showing measurement curves according to FIG. 6 for a respiratory minute volume of 100 L.

### DESCRIPTION OF THE PREFERRED EMBODIMENTS

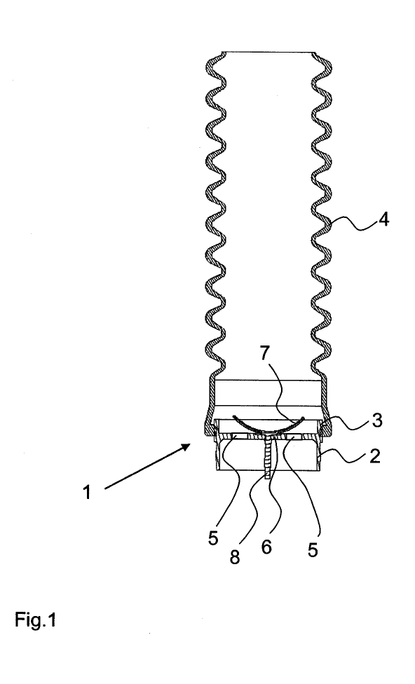

Referring to the drawings in particular, FIG. 1 shows a longitudinal section of a first directional valve according to the state of the art. A first valve housing 2 is connected to a breathing tube 4 in the outer area 3. The valve housing 2 has a flat contact surface 6 provided with holes 5 for a closing element 7, which is attached to a web 8 arranged in the center. In the flow direction of the first directional valve 1 shown in FIG. 1, the closing element 7 lifts up from the contact surface 6 and a gas flow through the holes 5 is possible. In the locking direction, the closing element 7 lies on the contact surface 6 and closes the holes 5.

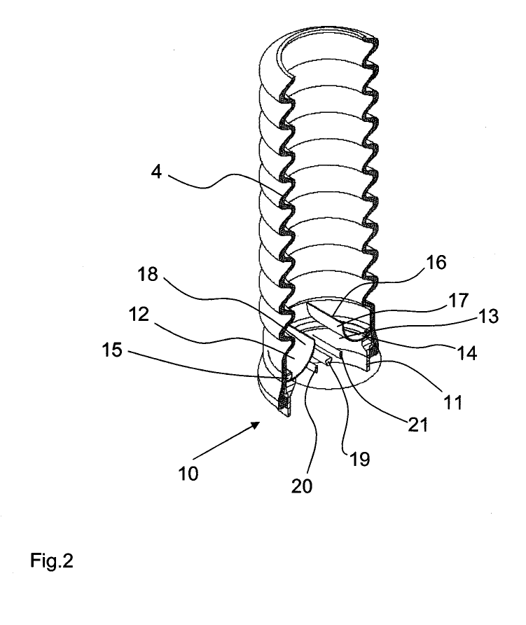

FIG. 2 illustrates a longitudinal section of a second directional valve 10 according to the present invention. A second, ring-shaped valve housing 11 is connected to the breathing tube 4. Two semicircular valve disks 12, 13 are attached to the second valve housing 11 in such a way that they, starting from a fixed section 14, 15 at the second valve housing 11, have a movable, second section 17, 18, running towards a common line of separation 16. The second valve housing 11 is provided with a central web 19 running along the line of separation 16, which serves as the valve seat for the valve disks 12, 13, wherein additional support webs 20, 21 are located on both sides of the central web 19.

FIG. 2 shows the second directional valve 10 in the flow direction, in which the valve disks 12, 13 lift up in a flap-like manner from the webs 19, 20, 21. In the locking direction the valve disks 12, 13 lie on the webs 19, 20, 21.

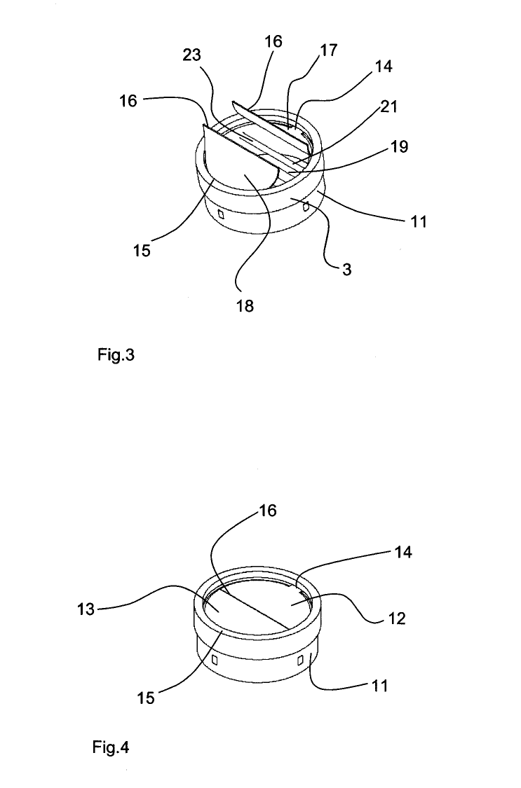

FIG. 3 shows a perspective view of the second directional valve corresponding to FIG. 2 in the flow direction. By contrast, FIG. 4 shows the locking direction of the second directional valve. Identical components are provided with the same reference numbers of FIG. 2. The outer area 3 of the second valve housing 11 is used for attaching the valve disks 12, 13 to the fixed sections 14, 15, while the inner area 23 of the second valve housing 11 is covered by the valve disks 12, 13.

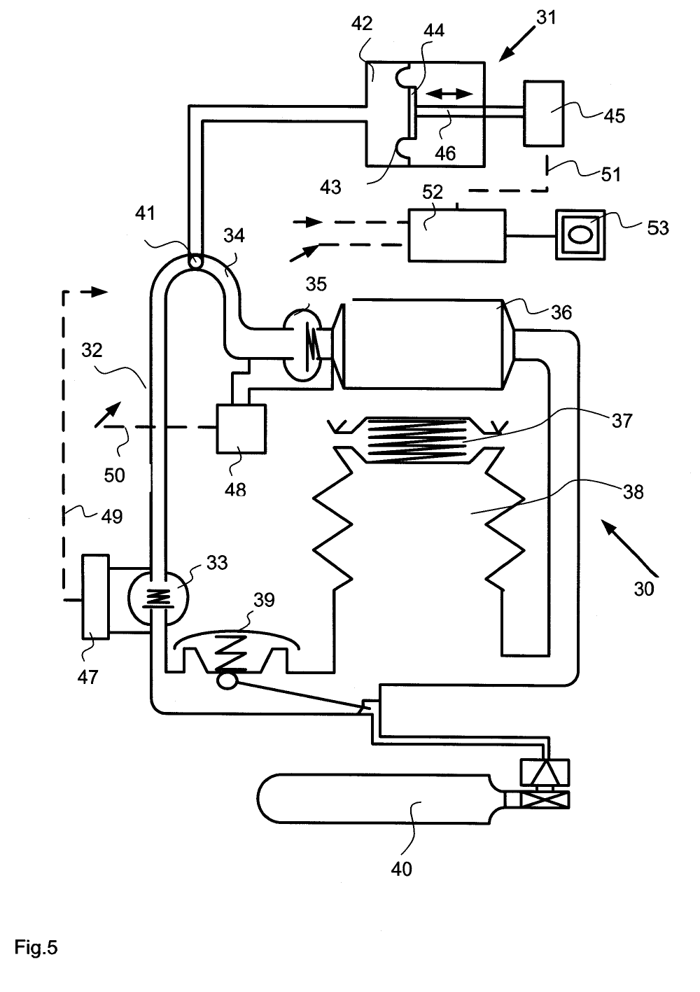

FIG. 5 schematically shows an arrangement for testing a respirator, which consists of a closed-circuit respirator 30 and a reciprocating pump 31. The closed-circuit respirator 30 comprises an inhalation tube 32 with an inhalation valve 33, an exhalation tube 34 with an exhalation valve 35, a regeneration cartridge 36 for the absorption of carbon dioxide, a breathing bag 38 loaded by a spring 37 and a demand oxygen system 39 with a pressurized gas source 40. The inhalation tube 32 and the exhalation tube 34 are connected to one another at a breathing connection 41, and the connection is made via the breathing connection 41 to a pressure space 42 of the reciprocating pump 31. The pressure space 42 of the reciprocating pump 31 is defined by an elastomer diaphragm 43 with a piston 44, whereby breaths are produced by means of a drive 45, which is connected via a push rod 46 to the piston 44.

A first pressure pickup 47 determines the differential pressure ΔP1 via the inhalation valve 33, and a second pressure pickup 48 determines the differential pressure ΔP2 via the exhalation valve 35. The pressure pickups 47, 48 and the drive 45 are connected via data lines 49, 50, 51 to a control unit 52, which controls the testing and issues measured values via a display unit 53.

The demand oxygen system 39, which replaces the consumed breathing gas during the normal use of the device, serves only for replacing the gas loss due to leaks during the testing.

A certain excess pressure is produced within the breathing circuit of the closed-circuit respirator 30 by the spring 37 which presses on the breathing bag 38. During exhalation, the breathing gas flows from the breathing connection 41 via the exhalation tube 34, the exhalation valve 35 and the regeneration cartridge 36 into the breathing bag 38 as storage volume. During inhalation, the breathing gas arrives from the breathing bag 38 and the inhalation valve 33 into the inhalation tube 32 and to the breathing connection 41.

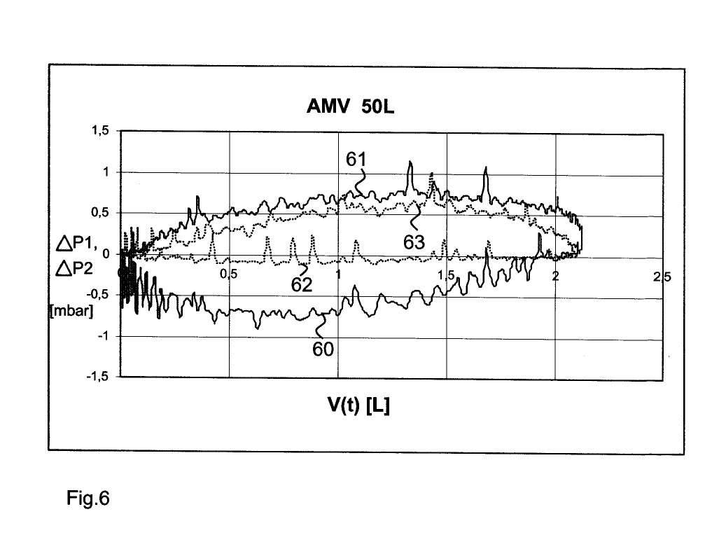

Measurement results with directional valves according to the state of the art according to FIG. 1 and directional valves according to the present invention according to FIG. 2 are compared in FIG. 6. The testing was performed with a respiratory minute volume of 50 L, corresponding to 25 strokes per minute with the reciprocating pump 31 and a stroke volume VT of 2 L.

FIG. 6 shows pressure measurement curves for a complete breathing cycle each, consisting of inhalation stroke and exhalation stroke. The time course of the breath V(t) with the maximum value VT is shown on the abscissa and the measured pressure differences ΔP1 and ΔP2 are shown on the ordinate. The measurement curves 60 and 61 illustrate the pressure courses in a directional valve according to FIG. 1. Curve 60 shows the pressure course ΔP1 for the inhalation valve 33 in the inhalation phase and curve 61 shows the pressure course ΔP2 for the exhalation valve 35 during the exhalation phase. During the inhalation phase the breathing gas is removed from the breathing bag 38, and the breathing resistance of the inhalation valve 33 must be overcome, which causes a certain inhalation effort. In the exhalation phase according to curve 61 for ΔP2, a rise in pressure is shown, since, in addition to the exhalation valve 35, the resistance of the regeneration cartridge 36 must be overcome, and the breathing bag 38 is filled against the force of the spring 37.

Curve 62 illustrates the pressure course ΔP1 during the inhalation phase for a directional valve according to the present invention according to FIG. 2. A marked reduction in the inhalation effort can be seen compared to curve 60. During the exhalation according to curve 63 and the pressure ΔP2, only the system-related flow resistances, caused by the regeneration cartridge 36 and the breathing bag 38 loaded by the spring 37, have to be overcome.

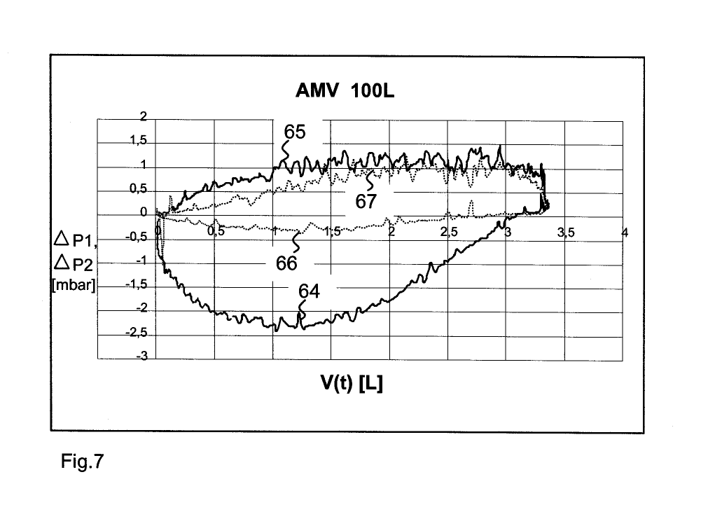

FIG. 7 shows measurement results for a respiratory minute volume of approximately 100 L corresponding to 29 strokes per minute with a stroke volume of 3.5 L. The curves 64, 65 show the pressure courses ΔP1 and ΔP2 for a directional valve according to FIG. 2. During the inhalation phase with the pressure course ΔP1, the directional valve according to FIG. 2, represented by the curve 66, shows a significantly lower flow resistance than the directional valve according to FIG. 1, with the curve 64.

While specific embodiments of the invention have been described in detail to illustrate the application of the principles of the invention, it will be understood that the invention may be embodied otherwise without departing from such principles.

### LIST OF REFERENCE NUMBERS

1 First directional valve
2 First valve housing
3 Outer area
4 Breathing tube
5 Hole
6 Contact surface
7 Closing element
8 Web
10 Second directional valve
11 Second valve housing
12, 13 Valve disk
14, 15 Fixed section
16 Line of separation
17, 18 Second section
19 Central web
20, 21 Support web
23 Inner area
30 Closed-circuit respirator
31 Reciprocating pump
32 Inhalation tube
33 Inhalation valve
34 Exhalation tube
35 Exhalation valve
36 Regeneration cartridge
37 Spring
38 Breathing bag
39 Demand oxygen system
40 Pressurized gas source
41 Breathing connection
42 Pressure space
43 Elastomer diaphragm
44 Piston
45 Drive
46 Push rod
47 First pressure pickup
48 Second pressure pickup
49, 50, 51 Data lines
52 Control unit
53 Display unit
60, 61, 62, 63, 64, 65, 66, 67 Measurement curve

What is claimed is:

1. A directional valve for a respirator product, the directional valve comprising:
an inner area;
an outer area;
a ring-shaped valve housing in the outer area; and
two diaphragm-like valve disks abutting against one another at a line of separation, the valve disks each having a first section fixed at the valve housing and a movable second section running towards the line of separation, wherein the valve housing has a central web as a valve seat running along the line of separation and support webs arranged on both sides of the central web such that the valve disks rest on the central web and rest on the support webs in a locking direction and are removed from the central web and the support webs in a flap-like manner by the breathing gas stream in a passing direction.

2. A directional valve in accordance with claim 1, wherein the valve disks are disk-shaped and consist essentially of one or more of rubber, elastomer material, and silicone rubber.

3. A directional valve in accordance with claim 1, wherein the valve disks have an average thickness between 0.6 mm and 1.2 mm.

4. A directional valve in accordance with claim 2, wherein the valve disks have an average thickness between 0.6 mm and 1.2 mm.

5. A directional valve in accordance with claim 1, wherein an inside diameter of the valve housing is between 35 mm and 50 mm.

6. A directional valve in accordance with claim 2, wherein an inside diameter of the valve housing is between 35 mm and 50 mm.

7. A directional valve in accordance with claim 3, wherein an inside diameter of the valve housing is between 35 mm and 50 mm.

8. A directional valve in accordance with claim 1, wherein the valve disks have a Shore hardness of 20° Sha to 30° Sha.

9. A directional valve in accordance with claim 2, wherein the valve disks have a Shore hardness of 20° Sha to 30° Sha.

10. A directional valve in accordance with claim 3, wherein the valve disks have a Shore hardness of 20° Sha to 30° Sha.

11. A directional valve comprising:
a valve housing defining an interior through which gas passes in a passing direction, the valve housing having a plurality of webs extending across the interior through which gas passes and defining a valve seat; and
valve disks abutting against one another at a line of separation, the valve disks each having a first section fixed at the valve housing and a movable second section extending from the valve housing towards the line of separation, wherein said valve disks rest on the webs in a closed direction and are spaced away from the webs by the gas flowing in the passing direction.

12. A directional valve in accordance with claim 11, wherein the valve disks are disk-shaped and consist essentially of one or more of rubber, elastomer material, and silicone rubber.

13. A directional valve in accordance with claim 11, wherein the valve disks have an average thickness between 0.6 mm and 1.2 mm.

14. A directional valve in accordance with claim 12, wherein the valve disks have an average thickness between 0.6 mm and 1.2 mm.

15. A directional valve in accordance with claim 11, wherein an inside diameter of the valve housing is between 35 mm and 50 mm.

16. A directional valve in accordance with claim 12, wherein an inside diameter of the valve housing is between 35 mm and 50 mm.

17. A directional valve in accordance with claim 13, wherein an inside diameter of the valve housing is between 35 mm and 50 mm.

18. A directional valve in accordance with claim 11, wherein the valve disks have a Shore hardness of 20° Sha to 30° Sha.

19. A directional valve in accordance with claim 12, wherein the valve disks have a Shore hardness of 20° Sha to 30° Sha.

20. A directional valve in accordance with claim 13, wherein the valve disks have a Shore hardness of 20° Sha to 30° Sha.
